(19) United States
(12) Patent Application Publication
(10) Pub. No.: US 2008/0200662 A1
(43) Pub. Date: Aug. 21, 2008

(54) 9-Fatty Acid Hydroperoxide Lyase Genes

(75) Inventors: Hisao Kuroda, Shizuoka (JP); Hirotaka Kaneda, Shizuoka (JP); Toshiyuki Ooshima, Shizuoka (JP)

Correspondence Address: OBLON, SPIVAK, MCCLELLAND MAIER & NEUSTADT, P.C., 1940 DUKE STREET, ALEXANDRIA, VA, 22314, US

(73) Assignee: SAPPORO BREWERIES LIMITED, Shibuya-ku (JP)

(21) Appl. No.: 11/628,076

(22) PCT Filed: May 27, 2005
(86) PCT No.: PCT/JP05/09757

§ 371 Date: Jan. 4, 2007

(30) Foreign Application Priority Data
May 31, 2004 (JP) 2004-162138
Nov. 1, 2004 (JP) 2004-318233
Dec. 20, 2004 (JP) 2004-368306

(51) Int. Cl. C07H 21/00

(52) U.S. Cl. 536/23.2

(57) ABSTRACT

A 9-fatty acid hydroperoxide lyase gene consisting of the nucleotide sequence as set forth in SEQ ID No: 1, and a 9-fatty acid hydroperoxide lyase gene consisting of the nucleotide sequence as set forth in SEQ ID No: 5.

### TECHNICAL FIELD

The present invention relates to 9-fatty acid hydroperoxide lyase genes.

### BACKGROUND ART

Cereals used as food and food raw materials contain lipids and fatty acids. When the cereals are processed or preserved, these lipids and fatty acids are oxidized by autoxidation due to the lipoxygenase contained in the cereals, or by oxygen in the air, and produce lipid hydroperoxides and fatty acid hydroperoxides. These hydroperoxides then produce aldehydes by pyrolysis, chemical degradation and enzymatic degradation, and since the produced aldehydes have a disagreeable odor or fatty acid odor, the flavor of the cereals is considerably impaired thereby (Non-patent Document 1).

Among these aldehydes, trans-2-nonenal is known to be an aldehyde with a low odor threshold, and is thought to be responsible for the disagreeable odor of old beer which has been kept for a long time (Non-patent Document 2), or rice which has been kept for a long time that has suffered quality deterioration due to processing or storage (Non-patent Document 3 or 4).

The production of trans-2-nonenal in beer may for example occur according to the following mechanism (Non-patent Document 5). In beer brewing, there is a brewing process which saccharifies malt to yield wort. In this process, linoleic acid 9-hydroperoxide, a kind of fatty acid hydroperoxide, is produced. The produced linoleic acid 9-hydroperoxide undergoes cleavage by the 9-fatty acid hydroperoxide lyase in the beer raw materials such as malt, thereby producing trans-2-nonenal.

Aldehyde synthesis in plants by oxylipin synthesis system has been studied in detail for dicotyledons (Non-patent Document 6). Aldehydes are produced by a cascade reaction between lipoxygenase and fatty acid hydroperoxide lyase, and it is thought that the fatty acid hydroperoxide lyase is an enzyme which catalyzes the rate-determining step of this reaction system. It has been determined that among these various aldehydes, trans-2-nonenal is produced by catalysis of a cleavage reaction of linoleic acid 9-hydroperoxide by 9-fatty acid hydroperoxide lyase (Non-patent Document 7), and the structure of the linoleic acid 9-hydroperoxide lyase gene has also been clarified (Non-patent Document 8).

There has been little research on monocotyledons such as rice and wheat, and a fatty acid hydroperoxide lyase gene have been isolated only in barley. However, this fatty acid hydroperoxide lyase cannot use linoleic acid 9-hydroperoxide as a substrate, and therefore it does not participate in the production of trans-2-nonenal at all (Non-patent Document 9).

[Non-patent Document 1]; J. Bruce German, Food Processing and Lipid Oxidation. Impact of Processing on Food Safety Kluwer Academic/Plenum Publishers, New York, 1999.

[Non-patent Document 2]: Drost, B. W. et al, Flavor stability, J. Am. Soc. Brew. Chem., 48, 124-131. 1990.

[Non-patent Document 3]: Suzuki et al, Volatile components in stored rice (Oryza sativa (L.)) of varieties with and without lipoxygenase-3 in seeds. J. Agric. Food Chem., 47: 1119, 1999.

[Non-patent Document 4]: Lam. H. S. and Proctor A., Milled Rice Oxidation Volatiles and Odor Development. Journal of Food Science, 68; 2676-2681, 2003.

[Non-patent Document 5]: Kuroda et al, Characterization of factors involved in the production of 2(E)-nonenal during mashing, Biosci. Biotechnol. Biochem., 67: 691-697, 2003.

[Non-patent Document 6]: Blee, E., Phytooxylipins and plant defense reactions, Prog. Lipid Res., 37, 33-72, 1998.

[Non-patent Document 7]: Matsui, et al, Fatty acid 9- and 13-hydroperoxide lyases from cucumber, FEBS Lett. 481, 183-8, 2000.

[Non-patent Document 8]: Matsui, K. et al, Bell pepper fruit fatty acid hydroperoxide lyase is a cytochrome P450 (CYP74B) FEBS Lett., 394, 21-24, 1996.

[Non-patent Document 9]: Koeduka et al, Kinetics of barley FA hydroperoxide lyase are modulated by salts and detergents, Lipids, 38:1167-1172, 2003.

### DISCLOSURE OF THE INVENTION

### Problems to be Solved by the Invention

If the 9-fatty acid hydroperoxide lyase gene of monocotyledons were elucidated, a restriction fragment length polymorphism (RFLP) and single nucleotide polymorphism (SNP) could then be found, and it would be possible to select a strain from which 9-fatty acid hydroperoxide lyase had been eliminated, or a strain wherein the stability and activity of 9-fatty acid hydroperoxide lyase was low. It would also be possible to produce a strain in which 9-fatty acid hydroperoxide lyase activity was suppressed by a gene suppressing technique such as RNA interference (RNAi).

It is therefore an object of the invention to elucidate a 9-fatty acid hydroperoxide lyase gene of monocotyledons.

### Means for Solving the Problems

In recent years, although a large amount of genetic information has been collected by genome techniques, and it is possible to extract similar genes based on existing genetic information, it is very difficult to identify a catalytic action or a product. Although fatty acid hydroperoxide lyase belongs to the cytochrome P450 family, this family consists of thousands of kinds of similar gene clusters, and it is impossible to estimate the function of an enzyme protein from the similarity of gene sequences.

The Inventors screened many genes similar to fatty acid hydroperoxide lyase based on published genetic information for rice, and as a result of expressing these genes and investigating their functions, discovered that a gene consisting of the nucleotide sequence as set forth in SEQ ID No: 1 and a gene consisting of the nucleotide sequence as set forth in SEQ ID No: 5 converted linoleic acid 9-hydroperoxide into trans-2-nonenal, and thus arrived at the present invention.

Specifically, the invention provides a 9-fatty acid hydroperoxide lyase gene consisting of the nucleotide sequence as set forth in SEQ ID No: 1. The invention further provides a 9-fatty acid hydroperoxide lyase gene which encodes 9-fatty acid hydroperoxide lyase consisting of the amino acid sequence as set forth in SEQ ID No: 2.

The invention further provides a 9-fatty acid hydroperoxide lyase gene consisting of the nucleotide sequence as set forth in SEQ ID No: 5. The invention also provides a 9-fatty acid hydroperoxide lyase gene which encodes 9-fatty acid hydroperoxide lyase consisting of the amino acid sequence as set forth in SEQ ID No: 6.

### Effect of the Invention

Taking the gene of the invention as an indicator, it is possible to select a strain of 9-fatty acid hydroperoxide lyase with low stability and activity. By controlling or modifying the expression of the gene of the invention, it is also possible to produce a strain of 9-fatty acid hydroperoxide lyase with low stability and activity. This means that food can be provided which keeps its flavor over time.

### BRIEF DESCRIPTION OF THE DRAWINGS

FIG. 1 is a phylogenetic tree relating to AK105964 and AK107161.

FIG. 2(a) is a total ion chromatogram of GC-MS of a reaction product of a protein obtained by expressing the gene consisting of the nucleotide sequence as set forth in SEQ ID No: 1, with linoleic acid 9-hydroperoxide. (b) is a total ion chromatogram of a negative control.

FIG. 3 is a mass spectrogram of peak 1.

FIG. 4 is a graph showing trans-2-nonenal produced by reaction of a protein obtained by expressing the nucleotide sequence as set forth in SEQ ID No: 1, with linoleic acid 9-hydroperoxide.

FIGS. 5(a), (b) and (c) are total ion chromatograms of GC-MS of reaction products of 13-HPOD, 9-HPOT and 13-HPOT and a protein obtained by expressing the gene consisting of the nucleotide sequence as set forth in SEQ ID No: 1, respectively.

### DESCRIPTION OF REFERENCE NUMERALS

1-trans-2-nonenal, 2-1-nonanol (internal standard).

### BEST MODE FOR CARRYING OUT THE INVENTION

One of the 9-fatty acid hydroperoxide lyase genes of the invention derived from rice consists of the nucleotide sequence as set forth in SEQ ID No: 1 of the Sequence Listing. The 9-fatty acid hydroperoxide lyase polypeptide encoded by this gene consists of the amino acid sequence as set forth in SEQ ID No: 2 of the Sequence Listing. Another 9-fatty acid hydroperoxide lyase gene of the invention derived from rice consists of the nucleotide sequence as set forth in SEQ ID No: 5 of the Sequence Listing. The 9-fatty acid hydroperoxide lyase polypeptide encoded by this gene consists of the amino acid sequence as set forth in SEQ ID No: 6 of the Sequence Listing. 9-fatty acid hydroperoxide lyase converts linoleic acid 9-hydroperoxide into trans-2-nonenal which is responsible for the aging odor. Therefore, by suppressing the expression of, or converting the gene of the invention, the flavor of cereals can be maintained, and the gene of the invention can be used for such a purpose.

For example, there is a method of using RNAi and antisense RNA to control the expression of the gene of the invention and maintain the flavor of cereals. It is also possible to produce cereals which maintain their flavor by producing a 9-fatty acid hydroperoxide lyase gene with decreased trans-2-nonenal production activity using site-specific mutagenesis etc., and introducing this gene into cereals. As the introduction method, the Agrobacterium method, protoplast method, PEG method, electroporation method, particle gun method or microinjection method may be used.

The gene of the invention can be obtained from rice (for example, strains such as Nipponbare) expressing this gene, using RT-PCR method. More specifically, the gene of the invention can be obtained by extracting RNA from the rice by AGPC method, or guanidine/cesium chloride ultracentrifugation method, etc., and performing PCR using the obtained RNA as a template.

A gene which encodes 9-fatty acid hydroperoxide lyase which is a mutant of the gene of the invention, can also be synthesized by site-specific mutagenesis. As a method of introducing a mutation into a nucleic acid, a known method such as Kunkel method or Gapped duplex method, may be mentioned. For example, the mutation can be introduced using a mutation introduction kit (Mutan-K or Mutan-G, TAKARA), or a LA PCR in vitro Mutagenesis series kit (TAKARA).

### EXAMPLES

### Example 1

Using barley 13-fatty acid hydroperoxide lyase (CAC82980), barley rice allene oxide synthetase (CAB86383, CAB86384) and rice allene oxide synthetase (Q8VZX5, Q940D7), which are functionally known genes, homologous proteins were extracted using the protein-protein BLAST program in the database of NCBI (National Center for Biotechnology Information) and KOME (Knowledge-Based Oryza Molecular Biological Encyclopedia), setting an E-value=e⁻¹⁰ as a threshold.

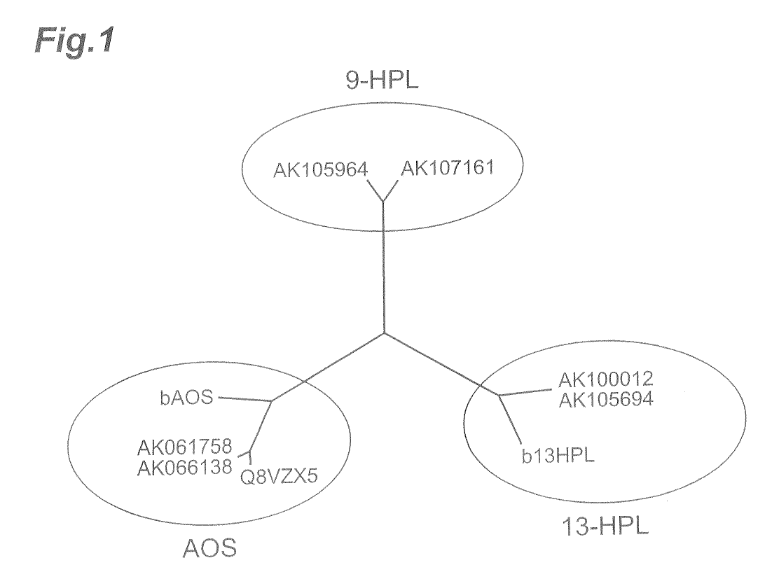

As a result, rice cDNAs AK105964 and AK107161 were newly extracted. When a molecular genealogical tree was created on an ExPASy Molecular Biology Server using ClastaIW, the molecular genealogical tree shown in FIG. 1 was obtained. As shown in FIG. 1, the two newly extracted genes were classified into clearly different groups from the allene oxide synthetase gene (AOS) and 13-fatty acid hydroperoxide lyase (13-HPL), and it was expected that they would have different enzyme functions from those of AOS and 13-HPL. Since AK105964 and AK107161 had very similar structures, a functional analysis was undertaken for AK105964.

### Example 2

After extraction of total RNA from rice (strain: Nipponbare) using a Tripure Isolation Reagent (Roche), PolyA⁺ RNA was purified using an Oligotex-dT30 mRNA Purification Kit (TAKARA Bio). RT-PCR was performed using two primers (5′-gcggatccatggcgcgccgccgcgagccaa-3′ (SEQ ID No: 3) and 5′gcgaattcccgcaccaacactcgccgctc-3′ (SEQ ID No: 4)), and subcloning into pUC118 was performed. The full length sequencing was performed, and it was confirmed that the sequence matched AK105964. Next, the structural gene of AK105964 was sub cloned between the BamHI site and EcoRI site of pRSETA (Invitrogen), and introduced into the E. coli strain BL21(DE3)pLys.

For expression of E. coli and preparation of a crude extract the method disclosed in Kuroda et al., Characterization of factors that transform linoleic acid into di- and trihydroxyoctadecenoic acids in mash, J. Biosci. Bioeng., 93: 73-77, 2002, was used. Specifically, isopropyl-β-thiogalactopyranoside was added to 50 ml of culture medium (OD600=0.5) pre-cultured at 16° C. so that the final concentration was 1 mM. Culture was continued for further 16 hours. E. coli cells were centrifuged and recovered, and suspended by adding 5 mL of Tris-HCl buffer solution (pH 7.2) containing 0.1% of Tween 20. After lysing the cells by sonication of the cell suspension and centrifuging for 30 minutes (15,000 g, 4° C.), the supernatant liquid was collected and taken as a crude extract. As a control sample, BL21(DE3)pLys into which pRSETA had been introduced was prepared, and a crude extract was obtained under the same conditions as those described above.

The activity measurement of fatty acid hydroperoxide lyase was performed according to the method disclosed in Kuroda et al., Characterization of factors involved in the production of 2(E)-noneal during mashing. Biosci. Biotechnol. Biochem., 67: 691-697, 2003. 10 mM Tris-HCl buffer solution (pH 7.2) containing 0.1% of Tween 20 was added to 5 μL of the crude extract and made up to 100 μL, then 5 μL of linoleic acid 9-hydroperoxide (2 mM) dissolved in ethanol was added, incubated for 5 minutes at 30° C. and the reaction was stopped by adding 900 μL of ethanol. The reactants were transferred to a vial to which 3 g of sodium chloride and 5 mL of distilled water had been added, 1 ppm of 1-nonanol (ethanol solution) was added as an internal standard, and the vial was capped.

Dosage and identification of compounds by gas chromatography-mass spectrometry (GC-MS) were performed as follows. A polydimethyl siloxane SPME fiber (Spelco) was inserted in the vial, and after incubating at 40° C. for 10 minutes, it was supplied to an injection port (260° C.) of a gas chromatography (Hewlett Packard HP6890/MSD system). The capillary column was DB-1 (30 m×0.25 mm, film thickness 1 μm, J&W Inc.), and the carrier gas was helium (1 mL/min). The oven conditions were 120° C. maintained for 1 minute, temperature increase up to 150° C. at 5° C./min, temperature increase up to 300° C. at 40° C./min, and temperature maintained at 300° C. for 2 minutes. The dosages of trans-2-nonenal and 1-nonanol were analyzed in the selected ion mode (m/z). The retention times of trans-2-nonenal and 1-nonanol were 7.6 minutes and 7.8 minutes, respectively. For GC-MS analysis, gas chromatography was performed as described above, and MS/MS analysis of the product and compound identification were performed using HP-Chemistation software and compound library.

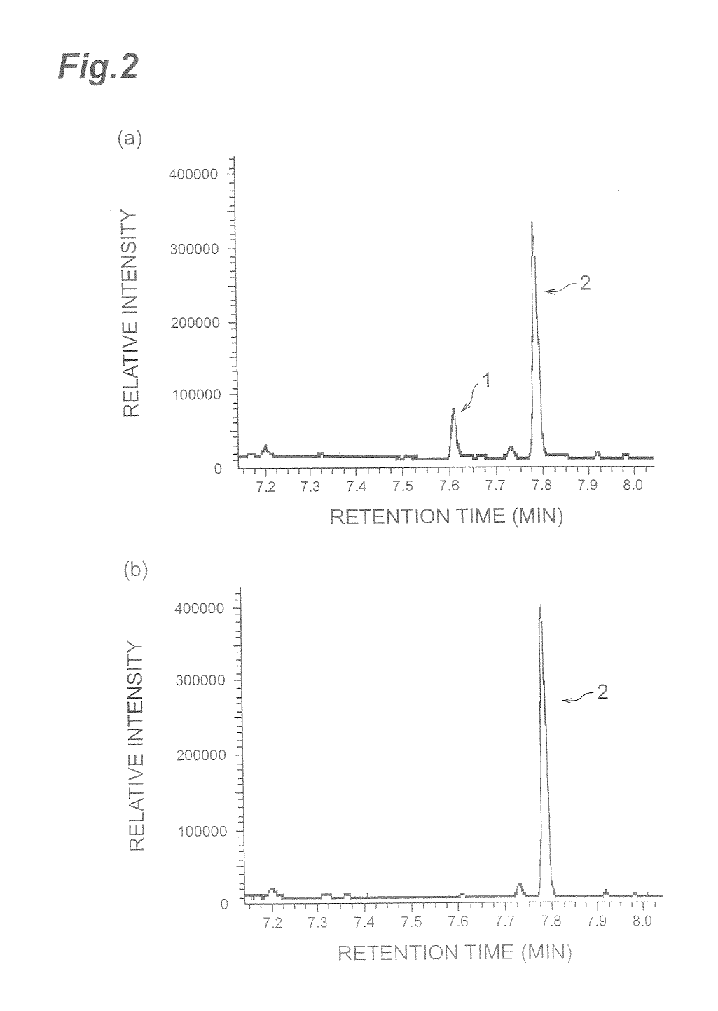

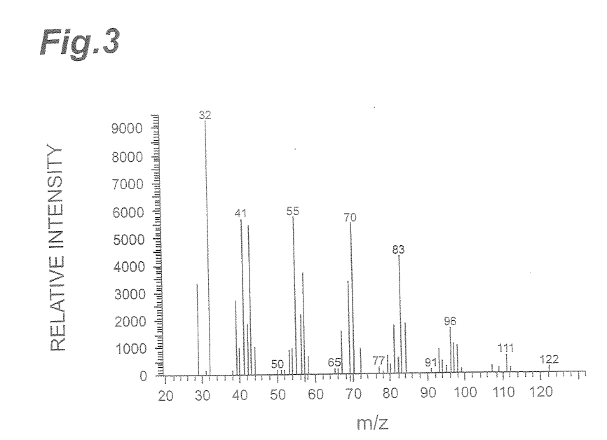

FIG. 2(a) is a diagram (total ion chromatogram) showing the GC-MS result for the reaction product of a protein obtained by expressing the gene consisting of the nucleotide sequence as set forth in SEQ ID No: 1 and linoleic acid 9-hydroperoxide. FIG. 2(b) shows a total ion chromatogram of a negative control. Peak 1 at a retention time of 7.6 minutes represents trans-2-nonenal, and peak 2 at a retention time of 7.8 minutes represents 1-nonanol, the internal standard. As shown in FIG. 2, the peak of trans-2-nonenal was observed only when the gene consisting of the nucleotide sequence as set forth in SEQ ID No: 1 was expressed. FIG. 3 shows the mass spectrogram of peak 1, and from this mass spectrogram, it is seen that peak 1 represents trans-2-nonenal.

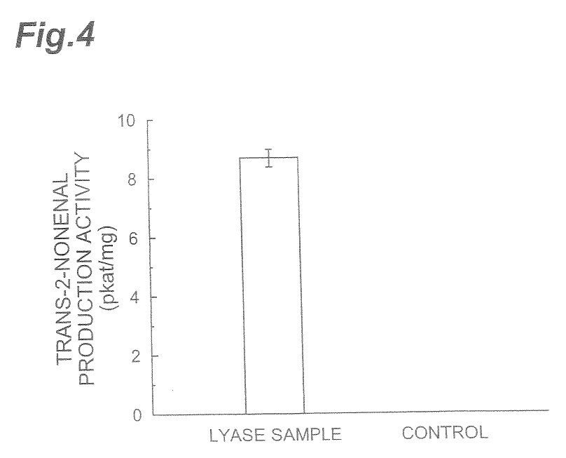

FIG. 4 is a graph showing trans-2-nonenal produced by reacting the protein obtained by expressing the gene consisting of the nucleotide sequence as set forth in SEQ ID No: 1 with linoleic acid 9-hydroperoxide. The vertical axis shows the activity of trans-2-nonenal per E. coli protein (mg). It is seen that trans-2-nonenal is produced only when the gene consisting of the nucleotide sequence as set forth in SEQ ID No: 1, is expressed.

An analysis was performed for AK107161 in an identical manner to that of AK105964. Using 5′-gccggatccatggcgccaccgccagt-3′ (SEQ ID No: 7) and 5′-gccgaattccgcaattaaaacatccaccaacct-3′ (SEQ ID No: 8) as primers, subcloning into pUC118 was performed. The full length sequencing was performed, and it was confirmed that the sequence matched AK107161. Next, the structural gene of AK107161 was subcloned between the BamHI site and EcoRI site of pRSETA, and introduced into E. coli strain BL21(DE3)pLys. Thereafter, it was expressed in E. coli by an identical method to that for AK105964, a crude extract was prepared, and it was found that AK107161 had an identical activity to that of AK105964.

From the above results, it was clear that the gene consisting of the nucleotide sequence as set forth in SEQ ID No: 1 and the gene consisting of the nucleotide sequence as set forth in SEQ ID No: 5, were 9-fatty acid hydroperoxide lyase genes which convert linoleic acid 9-hydroperoxide into trans-2-nonenal.

### Example 3

Next, the catalytic activities of the following fatty acid peroxides of AK105964 (and AK107161) (all peroxides were contained in rice), were analyzed: 13(S)-hydroperoxy-9(Z),11(E)-octadecadienoic acid (13-HPOD), 9(S)-hydroperoxy-10(E),12(Z),15(Z)-octadecatrienoic acid (9-HPOT) and 13(S)-hydroperoxy-9(Z),11(E)15(Z-octadecatrienoic acid (13-HPOT). As in Example 2, an enzyme extract and the fatty acid peroxide were reacted together, and the obtained product was analyzed by GC-MS.

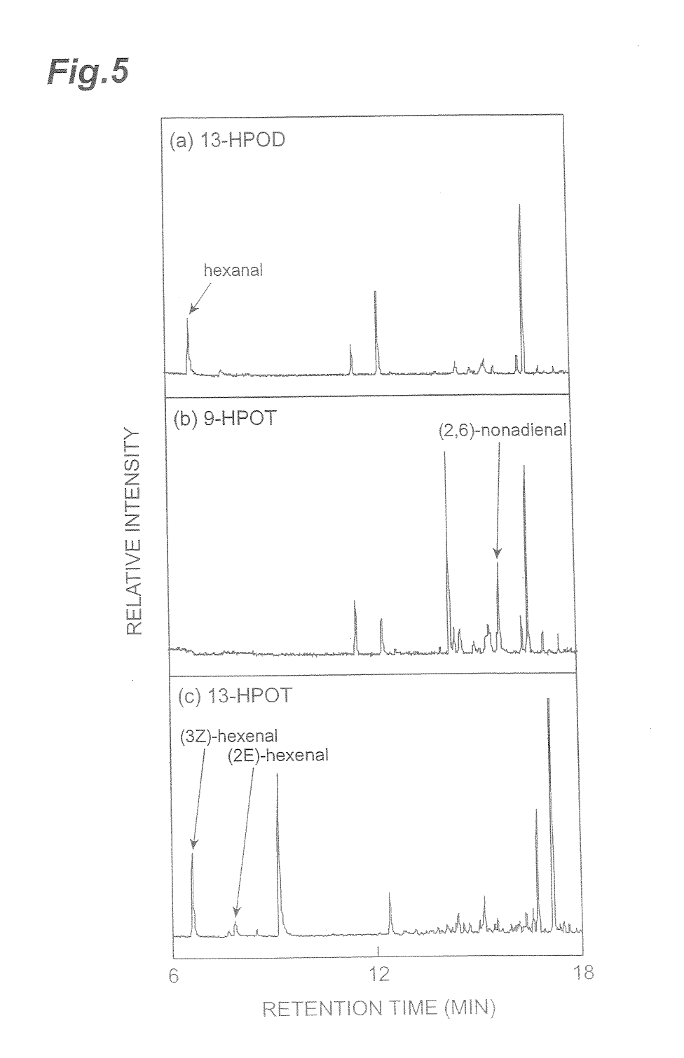

The obtained chromatograms are shown in FIGS. 5. (a), (b) and (c) are GC-MS chromatograms of the products obtained by reacting with 13-HPOD, 9-HPOT and 13-HPOT, respectively. As shown in FIGS. 5(a)-(c), hexanal was produced from 13-HPOD, (2,6)-nonadienal was produced from 9-HPOT, and (3Z)-hexenal and (2E)-hexenal was produced from 13-HPOT. The obtained aldehydes may be responsible for the off-flavor of foods, and it can be expected that the quality of rice will be improved by suppressing the expression of AK105964 (and AK107161) with gene suppressing techniques.

1. A 9-fatty acid hydroperoxide lyase gene consisting of the nucleotide sequence as set forth in SEQ ID No: 1.

2. A fatty acid hydroperoxide lyase gene encoding a 9-fatty acid hydroperoxide lyase consisting of the amino acid sequence as set forth in SEQ ID No: 2.

3. A 9-fatty acid hydroperoxide lyase gene comprising the nucleotide sequence as set forth in SEQ ID No: 5.

4. A fatty acid hydroperoxide lyase gene encoding a 9-fatty acid hydroperoxide lyase consisting of the amino acid sequence as set forth in SEQ ID No: 6.


SEQUENCE LISTING (8 sequences)

SEQ ID NO: 1 - DNA, 1533 bp, Oryza sativa
  CDS (1)..(1533)
atggcgccgccgcgagccaactccggcgacggtaacgacggcgccgtcggagggcagagcaagctctcgccgtcgggcctgctgatacgcgagattccgggcggctacggcgtgcccttcctctcgccgctgcgcgaccgcctcgactactattacttccagggcgccgacgagttcttccgctcacgcgtcgcccgccacggcggcgccaccgtgctccgcgtcaacatgccgcccggccccttcctcgccggcgacccccgcgtcgtcgccctcctcgacgcgcgcagcttccgcgtcctcctcgacgactccatggtggacaaggccgacacgctcgacggcaccttcatgccgtcgctcgcgctcttcggcggccaccgcccgctcgccttcctcgacgccgccgaccctcgccacgccaagatcaagcgcgtcgtgatgtcgctcgccgcggcgaggatgcaccacgtcgcgccggcgttccgcgccgccttcgccgccatgttcgacgaggtcgacgccggcctcgtcgccggcggccccgtcgagttcaacaagctcaacatgcggtacatgctcgacttcacctgcgccgcgctgttcggcggcgcgccgccgagcaaggccatgggcgacgctgccgtgacgaaggcggtgaagtggctcatcttccagcttcacccgctcgccagcaaggtcgtcaagccgtggccgctggaggacctcctcctccacaccttccgcctgccgccgttcctggtgcgccgcgagtacggcgagatcacggcgtacttcgccgccgccgccgcggccatcctcgacgacgccgagaagaaccacccgggaatcccgcgcgacgagctcctccacaacctcgtgttcgtcgccgtcttcaacgcctacggcggcttcaagatcttcctgccacacatcgtcaagtggctcgcccgcgccggcccggagctccacgccaagctagcctccgaggtccgcgccgccgcgcccgccggcggcggcgagatcaccatctccgccgtggagaaggagatgccgctggtgaagtcggtggtgtgggaggcgctgcgcatgaacccgccggtggagttccagtacgggcgcgcgcggcgcgacatggtcgtcgagagccacgacgcggcgtacgaggtccgcaagggggagctgctgttcgggtaccagccgctcgccacccgcgacgagaaggtgttcgaccgcgccggcgagttcgtccccgaccggttcgtctccggcgccggaagcgccgcccggccgctgctggagcacgtggtgtggtcgaacgggccggagaccgggacgccatcggaggggaacaagcagtgccccgggaaggacatggtggtggcggtggggcggctgatggtggcggggctgttccggcggtacgacacgttcgccgccgacgtggaggagctgccgcttgagccggtggtcacgttcacgtcgctgacccgcgccgccgacggcgacggcgccgcgcggcgcggagtataa

SEQ ID NO: 2 - protein, 510 aa, Oryza sativa
MAPPRANSGDGNDGAVGGQSKLSPSGLLIREIPGGYGVPFLSPLRDRLDYYYFQGADEFFRSRVARHGGATVLRVNMPPGPFLAGDPRVVALLDARSFRVLLDDSMVDKADTLDGTFMPSLALFGGHRPLAFLDAADPRHAKIKRVVMSLAAARMHHVAPAFRAAFAAMFDEVDAGLVAGGPVEFNKLNMRYMLDFTCAALFGGAPPSKAMGDAAVTKAVKWLIFQLHPLASKVVKPWPLEDLLLHTFRLPPFLVRREYGEITAYFAAAAAAILDDAEKNHPGIPRDELLHNLVFVAVFNAYGGFKIFLPHIVKWLARAGPELHAKLASEVRAAAPAGGGEITISAVEKEMPLVKSVVWEALRMNPPVEFQYGRARRDMVVESHDAAYEVRKGELLFGYQPLATRDEKVFDRAGEFVPDRFVSGAGSAARPLLEHVVWSNGPETGTPSEGNKQCPGKDMVVAVGRLMVAGLFRRYDTFAADVEELPLEPVVTFTSLTRAADGDGAARRGV

SEQ ID NO: 3 - DNA, 30 bp, Artificial Sequence
  Synthetic Oligonucleotide
gcggatccatggcgcgccgccgcgagccaa

SEQ ID NO: 4 - DNA, 29 bp, Artificial Sequence
  Synthetic Oligonucleotide
gcgaattcccgcaccaacactcgccgctc

SEQ ID NO: 5 - DNA, 1503 bp, Oryza sativa
  CDS (1)..(1503)
atggcgccaccgccagtgaactccggcgacgccgccgccgccgccacgggagagaagagcaagctctcgccgtcgggcctccccatacgcgagatacccggcggctacggcgtgcccttcttctcgccgctgcgcgaccgcctcgactacttctacttccagggcgccgaggagtacttccgatcacgcgtcgcccgccacggcggcgccaccgtgctccgcgtcaacatgccgcccggccccttcatctccggcaacccccgcgtcgtcgccctcctcgacgcgcgcagcttccgcgtcctcctcgacgactccatggtggacaaggccgacacgctcgacggcacctacatgccgtcgcgcgcgctcttcggcggccaccgcccgctcgccttcctcgacgccgccgacccgcgccacgccaagatcaagcgcgtcgtgatgtcgctcgccgccgcgcggatgcaccacgtcgcgccggcgttccgcgccgcctttgccgccatgttcgacgccgtcgaggccggcctcggcgccgccgtcgagttcaacaagctcaacatgaggtacatgctcgacttcacctgcgccgcgctgttcggcggcgagccgccgagcaaggtggtcggcgacggcgccgtgacgaaggccatggcgtggctcgcgttccagctgcacccgatcgcgagcaaggtcgtcaagccatggccgctcgaggagctactcctgcacaccttctccctgccgccgttcctggtgcggcgtggctacgccgacctgaaggcgtacttcgccgacgccgccgcggccgtcctcgacgacgccgagaagagccacacgggaatcccgcgcgacgagctcctcgacaaccttgtgttcgtcgccattttcaacgccttcggcggcttcaagatcttcctgccacacatcgtcaagtggctcgcccgcgccggcccggagctccacgccaagcttgccaccgaggtccgcgccaccgtgcccaccggcgaggacgacggcatcaccctcgccgccgtcgagcggatgccgctggtgaagtcggtggtgtgggaggcgctgcgcatgaacccgccggtggagttccagtacggccacgcgcggcgcgacatggtggtcgagagccacgacgcggcgtacgaggtgcgcaagggggagatgctgttcggctaccagccgctcgccacccgcgacgagaaggtgttcgaccgcgccggcgagttcgtcgccgaccggttcgtcgccggcggcgccgccggcgaccggccgctgctggagcacgtggtgtggtcgaacgggccggagacgagggcgccatcggaggggaacaagcagtgccccgggaaggacatggtggtggcggtggggcggctgatggtggcggagctgttccggcggtacgacacgttcgccgccgacgtggtggaggcgccggtggagccggtggtgacgttcacgtcgctgacacgggcgtcgtcgggatag

SEQ ID NO: 6 - protein, 500 aa, Oryza sativa
MAPPPVNSGDAAAAATGEKSKLSPSGLPIREIPGGYGVPFFSPLRDRLDYFYFQGAEEYFRSRVARHGGATVLRVNMPPGPFISGNPRVVALLDARSFRVLLDDSMVDKADTLDGTYMPSRALFGGHRPLAFLDAADPRHAKIKRVVMSLAAARMHHVAPAFRAAFAAMFDAVEAGLGAAVEFNKLNMRYMLDFTCAALFGGEPPSKVVGDGAVTKAMAWLAFQLHPIASKVVKPWPLEELLLHTFSLPPFLVRRGYADLKAYFADAAAAVLDDAEKSHTGIPRDELLDNLVFVAIFNAFGGFKIFLPHIVKWLARAGPELHAKLATEVRATVPTGEDDGITLAAVERMPLVKSVVWEALRMNPPVEFQYGHARRDMVVESHDAAYEVRKGEMLFGYQPLATRDEKVFDRAGEFVADRFVAGGAAGDRPLLEHVVWSNGPETRAPSEGNKQCPGKDMVVAVGRLMVAELFRRYDTFAADVVEAPVEPVVTFTSLTRASSG

SEQ ID NO: 7 - DNA, 26 bp, Artificial Sequence
  Synthetic Oligonucleotide
gccggatccatggcgccaccgccagt

SEQ ID NO: 8 - DNA, 33 bp, Artificial Sequence
  Synthetic Oligonucleotide
gccgaattccgcaattaaaacatccaccaacct